(19) United States
(12) Patent Application Publication
(10) Pub. No.: US 2011/0136913 A1
(43) Pub. Date: Jun. 9, 2011

(54) ASSESSING AND TREATING BREAST CANCER PATIENTS

(76) Inventors: Matthew P. Goetz, Rochester, MN (US); James N. Ingle, Rochester, MN (US); Vera J. Suman, Saint Charles, MN (US); Matthew M. Ames, Rochester, MN (US); Carol Reynolds, Rochester, MN (US)

(21) Appl. No.: 13/059,590

(22) PCT Filed: Aug. 19, 2009
(86) PCT No.: PCT/US09/54296

§ 371 Date: Feb. 17, 2011

(30) Foreign Application Priority Data
Aug. 19, 2008 (US) 61090131

(51) Int. Cl. A61K 31/138; C12Q 1/02; C12Q 1/68; G01N 33/574; A61P 35/00

(52) U.S. Cl. 514/648; 435/29; 435/6.11; 435/7.23

(57) ABSTRACT

This document provides methods and materials related to assessing and treating breast cancer patients. For example, methods and materials that involve assessing a breast cancer patient to determine whether the patient has (1) cancer cells that over express HER-2 polypeptides or comprise a HER-2 amplification and (2) at least an intermediate CYP2D6 metabolizer status are provided.

### CROSS-REFERENCE TO RELATED APPLICATIONS

This application claims the benefit of priority to U.S. Provisional Application Ser. No. 61/090,131, filed Aug. 19, 2008. The disclosure of the prior application is considered part of (and is incorporated by reference in) the disclosure of this application.

### STATEMENT AS TO FEDERALLY SPONSORED RESEARCH

This invention was made with government support under grants CA090628, CA116201, and CA025224 awarded by National Institutes of Health. The government has certain rights in the invention.

### BACKGROUND

1. Technical Field

This document relates to methods and materials involved in treating breast cancer patients and methods and materials involved in assessing breast cancer patients to aid in treatment decisions.

2. Background Information

In the adjuvant treatment of estrogen receptor (ER) positive breast cancer, hormonal therapy reduces the risk of breast cancer recurrence and decreases mortality. Tamoxifen, one of the most commonly used medications in the adjuvant treatment of ER positive breast cancer, is a selective ER modulator that competes with estrogen for binding to the ER. When administered to women with surgically treated ER positive breast cancer, tamoxifen reduces the risk of recurrence and death when taken for five years.

HER-2 gene amplification, when present in women with ER positive breast cancer, is associated with resistance to endocrine therapy, and specifically to tamoxifen (Carlomagno et al., J. Clin. Oncol., 14:2702-2708 (1996); Ellis et al., J. Clin. Oncol., 19:3808-3816 (2001); and Smith et al., J. Clin. Oncol., 23:5108-16 (2005)). This is thought to be due to ER/HER-2 cross talk, where tamoxifen binding to membrane bound ER stimulates the growth of tumor cells (Shou et al., J. Nat'l. Cancer Inst., 96:926-35 (2004)). This has been demonstrated in an in vivo mouse model using MCF-7 cell lines that stably over express exogenous HER-2 (Benz et al., Breast Cancer Res. Treat, 24(2):85-95 (1992)). In these cells, tamoxifen acts as a potent agonist on tumor growth and induces rapid (non-genomic) activation of EGFR/HER-2 signaling, which leads to activation of both p42/44 MAPK and AKT signal transduction pathways (Benz et al., Breast Cancer Res. Treat, 24(2):85-95 (1992) and Shou et al., J. Nat'l. Cancer Inst., 96:926-35 (2004)). Thus, it has been suggested that patients with ER positive breast cancer that over expresses HER-2 polypeptides should be preferentially recommended an aromatase inhibitor over tamoxifen (DePlacido et al., Clin. Can. Res., 9(3):1039-46 (2003)).

### SUMMARY

This document provides methods and materials related to assessing and treating breast cancer patients. For example, this document provides methods and materials that involve assessing a breast cancer patient to determine whether the patient has (1) cancer cells that over express HER-2 polypeptides and (2) at least an intermediate CYP2D6 metabolizer status. As described herein, breast cancer patients (e.g., ER positive breast cancer patients) who have cancer cells that over express HER-2 polypeptides and have at least an extensive CYP2D6 metabolizer status are likely to experience long disease free survival when treated with an endocrine cancer therapy (e.g., tamoxifen therapy). CYP2D6 metabolizer status refers to a patient's ability to metabolize an endocrine cancer therapy (e.g., tamoxifen) via cytochrome P450, family 2, subfamily D, polypeptide 6 (CYP2D6). A person can have a poor, intermediate, or extensive CYP2D6 metabolizer status as described herein. Patients with poor or intermediate CYP2D6 status are herein referred to as having decreased CYP2D6 metabolism.

In general, one aspect of this document features a method for assessing the likelihood of cancer treatment responsiveness, wherein said method comprises: (a) identifying a breast cancer patient having cancer cells over expressing HER-2 polypeptides or comprising a HER-2 amplification, thereby indicating that said breast cancer patient is a HER-2 over expressing breast cancer patient, and (b) determining that said HER-2 over expressing breast cancer patient comprises an extensive CYP2D6 metabolizer status, wherein the presence of said extensive CYP2D6 metabolizer status indicates that said HER-2 over expressing breast cancer patient is likely to respond to endocrine cancer therapy. The identifying step can comprise using an immunohistochemical assay for HER-2 polypeptide expression or a FISH assay. The determining step can comprise assessing the CYP2D6 genotype of the HER-2 over expressing breast cancer patient. The determining step can comprise determining that the HER-2 over expressing breast cancer patient is not receiving CYP2D6 inhibitory medication. The identifying step can be performed before the determining step. The determining step can be performed before the identifying step. The endocrine cancer therapy can comprise administering tamoxifen. The method can comprise administering tamoxifen after the HER-2 over expressing breast cancer patient is determined to comprise the extensive CYP2D6 metabolizer status.

In another embodiment, this document features a method for aiding in the treatment of breast cancer, wherein the method comprises: (a) identifying a breast cancer patient having cancer cells over expressing HER-2 polypeptides or comprising a HER-2 amplification, thereby indicating that the breast cancer patient is a HER-2 over expressing breast cancer patient, (b) determining that the HER-2 over expressing breast cancer patient comprises an extensive CYP2D6 metabolizer status, and (c) providing an indication that the HER-2 over expressing breast cancer patient is likely to be responsive to endocrine cancer therapy if the HER-2 over expressing breast cancer patient comprises the extensive CYP2D6 metabolizer status and providing an indication that the HER-2 over expressing breast cancer patient is unlikely to be responsive to endocrine cancer therapy if the HER-2 over expressing breast cancer patient does not comprise the extensive CYP2D6 metabolizer status. The identifying step can comprise using an immunohistochemical assay for HER-2 polypeptide expression or a FISH assay. The determining step can comprise assessing the CYP2D6 genotype of the HER-2 over expressing breast cancer patient. The determining step can comprise determining that the HER-2 over expressing breast cancer patient is not receiving CYP2D6 inhibitory medication. The identifying step can be performed before the determining step. The determining step can be performed before the identifying step. The endocrine cancer therapy can comprise administering tamoxifen. The method can comprise administering tamoxifen if the HER-2 over expressing breast cancer patient is determined to comprise the extensive CYP2D6 metabolizer status.

In another embodiment, this document features a method for treating breast cancer, wherein the method comprises administering an endocrine cancer therapy to a breast cancer patient after the breast cancer patient is determined to have (a) cancer cells over expressing HER 2 polypeptides or comprising a HER-2 amplification and (b) an extensive CYP2D6 metabolizer status. The breast cancer patient can be determined to have the cancer cells using an immunohistochemical assay for HER-2 polypeptide expression or a FISH assay. The breast cancer patient can be determined to have the extensive CYP2D6 metabolizer status by a method that comprises assessing the CYP2D6 genotype of the breast cancer patient. The breast cancer patient can be determined to have the extensive CYP2D6 metabolizer status by a method that comprises determining that the breast cancer patient is not receiving CYP2D6 inhibitory medication. Administering the endocrine cancer therapy can comprise administering tamoxifen.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides methods and materials related to assessing and treating women with breast cancer (e.g., ER positive breast cancer). For example, this document provides methods and materials that involve assessing a breast cancer patient to determine whether the patient has (1) cancer cells that over express or have amplification of HER-2 polypeptides and (2) an extensive CYP2D6 metabolizer status. As described herein, breast cancer patients (e.g., ER positive breast cancer patients) who have an extensive CYP2D6 metabolizer status and (a) have cancer cells that over express HER-2 polypeptides or (b) have an HER2 gene amplification are likely to experience long disease free survival when treated with an endocrine cancer therapy (e.g., tamoxifen therapy).

Any appropriate method can be used to determine whether breast cancer over expresses HER-2 polypeptides. For example, an immunohistochemical assay such as the HerceptTest (Dako HerceptTest, Carpinteria, Calif. 93013; code k5207) or a FISH analysis for HER2 amplification (e.g., PathVysion HER2 DNA probe kit) can be used to determine whether or not a patient has breast cancer cells that over express HER-2 polypeptides. The results of such assays can be scored using standard criteria such that a positive result (e.g., a 3+ result) indicates that the breast cancer cells over express HER-2 polypeptides and a negative result (e.g., a 0, 1, or 2 result) indicates that the breast cancer cells do not over express HER-2 polypeptides. See, e.g., Perez et al., J. Clin. Oncol., 24:3032-8 (2006)). In some cases, HER-2 FISH amplification can be scored such that a positive test A positive FISH result is defined as at least 2.0 HER-2 probe/control probe ratio (Perez et al., J. Clin. Oncol., 24:3032-8 (2006)).

CYP2D6 metabolizer status refers to a patient's ability to metabolize an endocrine cancer therapy (e.g., tamoxifen) via CYP2D6. A person can have a poor, intermediate, or extensive CYP2D6 metabolizer status as described herein. Any appropriate method can be used to determine whether or not a patient has a poor, intermediate, or the *3, *4, *5, *6, *10, *17, and *41 alleles. In some cases, a patient having a CYP2D6 *4/WT or a CYP2D6 *4/*4 genotype can be classified as having decreased CYP2D6 metabolizer status. In some cases, a patient without the *3, *4, or *6 alleles and who is heterozygous for *10, *17, or *41 alleles can be classified as having an extensive CYP2D6 metabolizer status. Patients homozygous for a intermediate CYP2D6 allele (e.g., a *10, *17, and *41 allele) can be referred to as CYP2D6 intermediate metabolizers as well as having decreased CYP2D6 metabolism. Any patients with either one intermediate allele in the presence of a null allele (referred to as *3, *4, *6) can also be referred to as having decreased CYP2D6 metabolism. Standard PCR and sequencing techniques can be used to determine a patients CYP2D6 genotype. See, e.g., Borges et al., Clin. Pharmacol. Ther., 80:61-74 (2006)).

In some cases, a patient having extensive CYP2D6 genotype can be classified as having decreased CYP2D6 metabolizer status during the period of time that the patient is taking a CYP2D6 inhibitor medication (Desta et al., J. Nat'l. Cancer Inst., 97:30-9 (2005)). Examples of CYP2D6 inhibitors include, without limitation, fluoxetine, paroxetine sertraline, cimetidine, amiodarone, doxepin, ticlopidine, and haloperidol. In some cases, a patient can be questioned to determine whether or not the patient is taking a CYP2D6 inhibitor.

The invention will be further described in the following examples, which do not limit the scope of the invention described in the claims.

### Example 1

### Assessing HER-2 Over Expression and CYP2D6 Metabolizer Status in Breast Cancer Patients

A retrospective analysis of women with ER positive breast cancer previously randomized to the tamoxifen-alone arm of an adjuvant tamoxifen trial (NCCTG 89-30-52) was performed. The North Central Cancer Treatment Group conducted a randomized phase III clinical trial in postmenopausal women with resected ER positive breast cancer to assess the value of adding one year of fluoxymesterone to 5 years of tamoxifen adjuvant therapy (NCCTG 89-30-52; Ingle et al., Breast Cancer Res. Treat., 98:217-22 (2006)). Postmenopausal women with node-negative disease were required to have stage T₁c or T₂N₀M₀ cancer and could be any age, whereas women with node-positive disease were required to be at least 65 years of age with a tumor stage T₁ (any N) or T₂N₁M₀. A woman was classified as postmenopausal if one of the following held: (1) her last menstrual cycle was >12 months prior to diagnosis, (2) her last menstrual cycle was 4-12 months prior to diagnosis and her follicle-stimulating hormone (FSH) level was in the postmenopausal range, (3) she had a bilateral oophorectomy at least 2 months prior to diagnosis, or (4) she had a hysterectomy without oophorectomy and was either >60 years old or her FSH level was in the postmenopausal range. Patients were surgically treated with either a modified radical mastectomy or breast conservative therapy including lumpectomy, axillary nodal dissection, and radiation therapy. The axillary dissection must have involved at least levels I and II and the examination of at least 6 axillary nodes. Patients who underwent lumpectomy must have had a primary tumor no larger than 5 cm, and the surgical margins must have been microscopically free of tumor. Postlumpectomy radiation therapy consisted of a total cumulative breast dose of 5040 cGy in 28 fractions, and those with axillary nodal involvement also received radiation to the axilla and supraclavicular regions. Patients were classified as ER positive if resected breast tissue contained ≧10 fmol of ER polypeptide/mg cytosol protein or was positive for ER polypeptide by an immunohistochemical assay. All patients were randomized within 6 weeks of definitive surgery.

CYP2D6 metabolism (extensive or decreased) was determined based on knowledge of CYP2D6**3, *4, *6, *10, *17, and *41 genotype and presence/absence of CYP2D6 inhibitor. HER-2 phenotype was determined using an immunohistochemical assay (HerceptTest) and was scored using the standard criteria as positive (3+), or negative (0, 1, 2) (Perez et al., J. Clin. Oncol., 24:3032-8 (2006)). Additionally, any sample that was negative for Hercept (0, 1, 2) was subjected for FISH analysis for HER2 (PathVysion (Vysis Inc, Downers Grove, Ill.)). If the HER2:CEP17 ratio was ≧2.0, the ratio was considered amplified (Perez et al., J. Clin. Oncol., 24:3032-8 (2006)).

CYP2D6 metabolizer status/HER-2 status representing the four categories of combining CYP2D6 metabolism (extensive or decreased) and HER-2 (positive or negative) was created. The association between CYP2D6:HER-2 and disease free survival and overall survival was assessed using the log-rank test and proportional hazards modeling.

Results. HER-2 evaluation for either IHC or FISH was available in 215 patients. HER-2 over expression was not associated with shorter disease free survival (HR 0.86, log rank p=0.616), In patients negative for HER2 (n=178), patients with impaired CYP2D6 metabolism had shorter DFS compared to patients with extensive CYP2D6 metabolism (HR 1.46; p=0.05). In patients positive for HER2 and in which there was CYP2D6 metabolism information (n=22), patients with decreased CYP2D6 metabolism had a 4.1 fold higher risk of disease recurrence compared to patients with normal CYP2D6 metabolism. At the 6 year mark, no patients positive for HER2 who were

CYP2D6 extensive metabolizers had documented recurrence or death. Conversely, 54% of patients with decreased CYP2D6 metabolism had recurred or died and by year 12, and 70% had recurred or died.

These results demonstrate that women who are CYP2D6 normal or extensive metabolizers, and have ER positive breast cancer that over expresses or is amplified for HER-2 can be treated with an endocrine cancer therapy (e.g., tamoxifen). These data also demonstrate that breast cancer patients that have evidence for HER2 amplification or over expression and who have decreased CYP2D6 metabolism have a much higher rate of recurrence.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing the likelihood of cancer treatment responsiveness, wherein said method comprises:
(a) identifying a breast cancer patient having cancer cells over expressing HER-2 polypeptides or comprising a HER-2 amplification, thereby indicating that said breast cancer patient is a HER-2 over expressing breast cancer patient, and
(b) determining that said HER-2 over expressing breast cancer patient comprises an extensive CYP2D6 metabolizer status,
wherein the presence of said extensive CYP2D6 metabolizer status indicates that said HER-2 over expressing breast cancer patient is likely to respond to endocrine cancer therapy.

2. The method of claim 1, wherein said identifying step comprises using an immunohistochemical assay for HER-2 polypeptide expression or a FISH assay.

3. The method of claim 1, wherein said determining step comprises assessing the CYP2D6 genotype of said HER-2 over expressing breast cancer patient.

4. The method of claim 1, wherein said determining step comprises determining that said HER-2 over expressing breast cancer patient is not receiving CYP2D6 inhibitory medication.

5. The method of claim 1, wherein said identifying step is performed before said determining step.

6. The method of claim 1, wherein said determining step is performed before said identifying step.

7. The method of claim 1, wherein said endocrine cancer therapy comprises administering tamoxifen.

8. The method of claim 1, wherein said method comprises administering tamoxifen after said HER-2 over expressing breast cancer patient is determined to comprise said extensive CYP2D6 metabolizer status.

9. A method for aiding in the treatment of breast cancer, wherein said method comprises:
(a) identifying a breast cancer patient having cancer cells over expressing HER-2 polypeptides or comprising a HER-2 amplification, thereby indicating that said breast cancer patient is a HER-2 over expressing breast cancer patient,
(b) determining that said HER-2 over expressing breast cancer patient comprises an extensive CYP2D6 metabolizer status, and
(c) providing an indication that said HER-2 over expressing breast cancer patient is likely to be responsive to endocrine cancer therapy if said HER-2 over expressing breast cancer patient comprises said extensive CYP2D6 metabolizer status and providing an indication that said HER-2 over expressing breast cancer patient is unlikely to be responsive to endocrine cancer therapy if said HER-2 over expressing breast cancer patient does not comprise said extensive CYP2D6 metabolizer status.

10. The method of claim 9, wherein said identifying step comprises using an immunohistochemical assay for HER-2 polypeptide expression or a FISH assay.

11. The method of claim 9, wherein said determining step comprises assessing the CYP2D6 genotype of said HER-2 over expressing breast cancer patient.

12. The method of claim 9, wherein said determining step comprises determining that said HER-2 over expressing breast cancer patient is not receiving CYP2D6 inhibitory medication.

13. The method of claim 9, wherein said identifying step is performed before said determining step.

14. The method of claim 9, wherein said determining step is performed before said identifying step.

15. The method of claim 9, wherein said endocrine cancer therapy comprises administering tamoxifen.

16. The method of claim 9, wherein said method comprises administering tamoxifen if said HER-2 over expressing breast cancer patient is determined to comprise said extensive CYP2D6 metabolizer status.

17. A method for treating breast cancer, wherein said method comprises administering an endocrine cancer therapy to a breast cancer patient after said breast cancer patient is determined to have (a) cancer cells over expressing HER-2 polypeptides or comprising a HER-2 amplification and (b) an extensive CYP2D6 metabolizer status.

18. The method of claim 17, wherein said breast cancer patient is determined to have said cancer cells using an immunohistochemical assay for HER-2 polypeptide expression or a FISH assay.

19. The method of claim 17, wherein said breast cancer patient is determined to have said extensive CYP2D6 metabolizer status by a method that comprises assessing the CYP2D6 genotype of said breast cancer patient.

20. The method of claim 17, wherein said breast cancer patient is determined to have said extensive CYP2D6 metabolizer status by a method that comprises determining that said breast cancer patient is not receiving CYP2D6 inhibitory medication.

21. The method of claim 17, wherein administering said endocrine cancer therapy comprises administering tamoxifen.
